(19) United States
(12) Patent Application Publication
(10) Pub. No.: US 2018/0279746 A1
(43) Pub. Date: Oct. 4, 2018

(54) HARNESS AND HOLDER FOR GAS DETECTOR

(71) Applicant: Ty-Flot, Inc., Manchester, NH (US)

(72) Inventors: Darrell A. Moreau, Manchester, NH (US); Andre W. Moreau, Bedford, NH (US)

(73) Assignee: Ty-Flot, Inc., Manchester, NH (US)

(21) Appl. No.: 15/471,381

(22) Filed: Mar. 28, 2017

(51) Int. Cl. A45F 3/14

(52) U.S. Cl. CPC A45F 3/14; G01N 33/0009; A45F 2003/146; A45F 2003/144

(57) ABSTRACT

A body harness has a waist belt configured to form a closed loop around a user's waist and has a side portion corresponding to the user's side and a rear portion corresponding to the user's back. A shoulder strap is constructed to extend over the user's shoulder and has a front portion with a first strap end connected to the side portion of the waist belt and a back portion with a second strap end connected to the rear portion of the waist belt. A side strap is connected to and extends between the front portion and the back portion of the shoulder strap. The side strap is vertically spaced from and extends generally parallel to the side portion of the waist belt. A pouch with an attachable probe pocket may be secured to the body harness.

### BACKGROUND OF THE INVENTION

### 1. Field of the Invention

The present invention relates generally to tool pouches and carrying cases. More particularly, the present invention relates to a body harness and tool pouch useful for a portable gas detector and like tools

### 2. Description of the Prior Art

In the regular course of a work day workers may be exposed to airborne contaminants that are harmful or fatal. Confined spaces in chemical plants, paper mills, refineries, underground mines, utility passageways, and other industrial locations may contain air that is contaminated with toxic or combustible gases or air depleted of oxygen. To protect workers, OSHA regulations require the worker to have a way to monitor the air prior to entry and during the entire time the worker occupies a confined space.

Each person entering a confined space should be equipped with a portable gas monitor worn where it can be checked easily and frequently. Typically, the gas detector has a sampling hose and probe attached to the gas monitor. An air sample is brought into the gas monitor by a sample pump. Upon detecting an unsafe condition, the gas monitor will transmit warnings via audible and visible signals, such as alarms and flashing lights.

When using a portable gas detector, the worker typically carries the gas detector in addition to the work tools and other equipment. One approach has been to place the gas monitor in a shoulder bag or holster on a tool belt. In other cases, the gas monitor has a shoulder strap and the worker carries the hose and probe in the hand.

### SUMMARY OF THE INVENTION

A problem with current equipment is that the gas detector presents a safety hazard itself. For example, when climbing or accessing confined spaces, a gas detector on a shoulder strap is able to swing free from the user's body and get tangled with or caught on the surrounding structures or equipment. Also, a gas monitor carried on a shoulder strap may become damaged when the worker moves about and the monitor makes contact with door frames, ladders, or other structures.

Therefore, what is needed is an improved apparatus for carrying a portable gas detector on one's person while on a jobsite and while retaining the ability to effectively and conveniently use the gas detector.

One aspect of the present invention is directed to a carrying pouch for a portable gas detector or other tool. In one embodiment, a tool pouch includes a support panel having a front face, a back face, a panel first side portion, a panel second side portion, and a panel bottom portion. A pouch body portion attached to the support panel has a first side portion, a second side portion spaced apart from and generally opposing the first side portion, and a bottom portion. The pouch body portion is attached to the support panel along the respective panel first side portion, the panel second side portion, and the panel bottom portion and extends out from the front face of the support panel to define a storage compartment. The pouch body portion defines an open region between the first side portion, the second side portion, and the bottom portion. One or more straps extend across the open region and connect the first side portion to the second side portion. A connector first portion is fixedly secured to the first side portion and/or the second side portion of the pouch body portion, where each connector first portion is positioned on an outside surface of the pouch body portion to releasably engage a corresponding connector second portion.

In some embodiments, each of the straps is convertible between a larger state and a smaller state. For example, each strap is made of a stretchable material.

In another embodiment, the pouch includes a probe pocket made of a pliable material and having a pocket sidewall with an elongated tubular shape extending from a closed bottom end to an open top end. The connector second portion is secured to the probe pocket adjacent the open top end, where the connector second portion is constructed to releasably engage the connector first portion to releasably attach the probe pocket to the main pouch body.

Another embodiment of a tool pouch has a main pouch body with a closed bottom end portion and a sidewall extending up from the closed bottom end portion to define a storage compartment. The sidewall includes a first side portion, a second side portion spaced apart from and generally opposing the first side portion, a front portion, and a rear portion spaced apart from and generally opposing the front portion. A connector first portion is fixedly secured to the sidewall and positioned on an outside surface of the pouch body. A probe pocket is made of a pliable material and has a pocket sidewall with an elongated tubular shape extending from a closed bottom end to an open top end. A connector second portion is secured to the probe pocket adjacent the open top end, where the connector second portion operably engages the connector first portion to releasably attach the probe pocket to the main pouch body.

In another embodiment, the front portion of the main pouch body defines an open region between the first side portion and the second side portion. In some embodiments, the tool pouch has one or more straps extending across the open region and connecting the first side portion to the second side portion. For example, the front portion of the sidewall is replaced substantially or completely by the open region and strap(s).

In some embodiments, each of the straps is convertible between a larger state and a smaller state, such as being made of a stretchable material.

In some embodiments, the pocket sidewall of the probe pocket defines a plurality of through openings. For example, the through openings are defined in a mesh material that allow gases to flow through the sidewall of the probe pocket to a sensor probe disposed in the probe pocket.

Another aspect of the present invention is directed to a body harness wearable by a user. For example, the body harness is configured to retain a pouch for a gas detector and optionally includes a separate pocket for a sensor probe.

In one embodiment, the body harness includes a waist belt configured to form a closed loop around a user's waist. The waist belt has a side portion corresponding to the user's side, a rear portion corresponding to the user's back, and a front portion with a closure. A shoulder strap is constructed to extend over a user's shoulder and has a front portion with a first strap end connected to the side portion of the waist belt and a back portion with a second strap end connected to the rear portion of the waist belt. A side strap is connected to and extends between the front portion and the back portion of the shoulder strap, where the side strap is vertically spaced from and extends generally parallel to the side portion of the waist belt.

In some embodiments of the body harness, a flexible cord-lock strap is secured to the body harness and has a releasable closure configured to releasably fasten the cord-lock strap in a closed loop. For example, the cord-lock strap is secured to the waist belt or to the front portion of the shoulder strap.

In another embodiment, the body harness includes one or more anchor point. For example, the anchor point includes a closed-loop connector, a releasable connector, or a cord loop.

In another embodiment, the body harness includes a tool pouch with a main pouch body, a connector first portion fixedly secured to the sidewall, and an attachment loop secured to the rear portion. For example, the main pouch body has a closed bottom end portion and a sidewall extending up from the closed bottom end portion to define a storage compartment. A connector first portion is fixedly secured to the sidewall and positioned on an outside surface of the pouch body. A selectively-closable attachment loop is secured to the rear portion and sized to extend through the closed-loop connector, the releasable connector, or the cord loop on the anchor point. The selectively-closable attachment loop may be, for example, a side-release buckle and webbing, a carabiner, or a length of webbing or cord with a fastener.

In some embodiments, the tool pouch includes a probe pocket made of a pliable material and having a pocket sidewall with an elongated tubular shape extending from a closed bottom end to an open top end. A connector second portion secured to the probe pocket adjacent the open top end is constructed to operably mate with the connector first portion to releasably secure the probe pocket to the main pouch body.

In some embodiments, the body harness includes a tool pouch releasably attached to the body harness, where the tool pouch includes a support panel having a front face, a back face, a panel first side portion, a panel second side portion, a panel bottom portion, and a pouch body portion attached to the support panel to define a storage compartment. The pouch body portion has a first side portion, a second side portion spaced apart from and generally opposing the first side portion, and a bottom portion. The pouch body portion is attached to the support panel along the respective panel first side portion, the panel second side portion, and the panel bottom portion and extends out from the front face of the support panel to define a storage compartment. The pouch body portion defines an open region between the first side portion, the second side portion, and the bottom portion. One or more straps extend across the open region and connect the first side portion to the second side portion. A connector first portion fixedly secured to one or both of the first and second side portions is positioned on an outside surface of the pouch body portion to releasably engage a corresponding connector second portion.

In some embodiments, each of the one or more straps is convertible between a larger state and a smaller state, such as being made of a stretchable material.

In some embodiments, the pocket sidewall of the probe pocket defines a plurality of through openings.

### BRIEF DESCRIPTION OF THE DRAWINGS

FIG. 1 is a front elevational view of one embodiment of a tool pouch assembly of the present invention configured for a gas detector and a sensor probe, where the pouch assembly includes a tool pouch and a probe pocket releasably attached to the tool pouch.

FIG. 2 is a rear elevational view of the tool pouch assembly of FIG. 1 showing a connector and webbing secured to the back surface of the tool pouch and defining a loop.

FIG. 3 is a front perspective view of a tool pouch assembly of the present invention showing a portable gas detector retained in the tool pouch, the sensor probe retained in the probe pocket, and a cord-lock strap installed around the tubing between the sensor probe and the gas detector.

FIG. 4 is a front perspective view of another embodiment of a tool pouch assembly of the present invention showing the tool pouch having a closed front portion and the probe pocket defining a plurality of through openings.

FIG. 5 is a front perspective view of another embodiment of a tool pouch assembly of the present invention with a portable gas detector and showing a first probe pocket fixedly attached by stitching to the tool pouch and a second probe pocket releasably attached to the tool pouch with a side-release buckle connector.

FIG. 6 is a front view showing one embodiment of a body harness of the present invention, where the body harness is worn by a user and includes a waist belt, a shoulder strap, and a side strap.

FIG. 7 is a rear view of the body harness of FIG. 4 showing the back portion of the shoulder strap and waist belt.

FIG. 8 is a front view of one embodiment of a body harness of the present invention in use by a user, where the body harness is configured with a tool pouch and probe pocket, and where the gas detector is installed in the tool pouch, the sensor probe is disposed in the probe pocket, and the gas tubing is retained by a cord-lock strap secured to the front portion of the shoulder strap.

### DETAILED DESCRIPTION OF THE PREFERRED EMBODIMENT

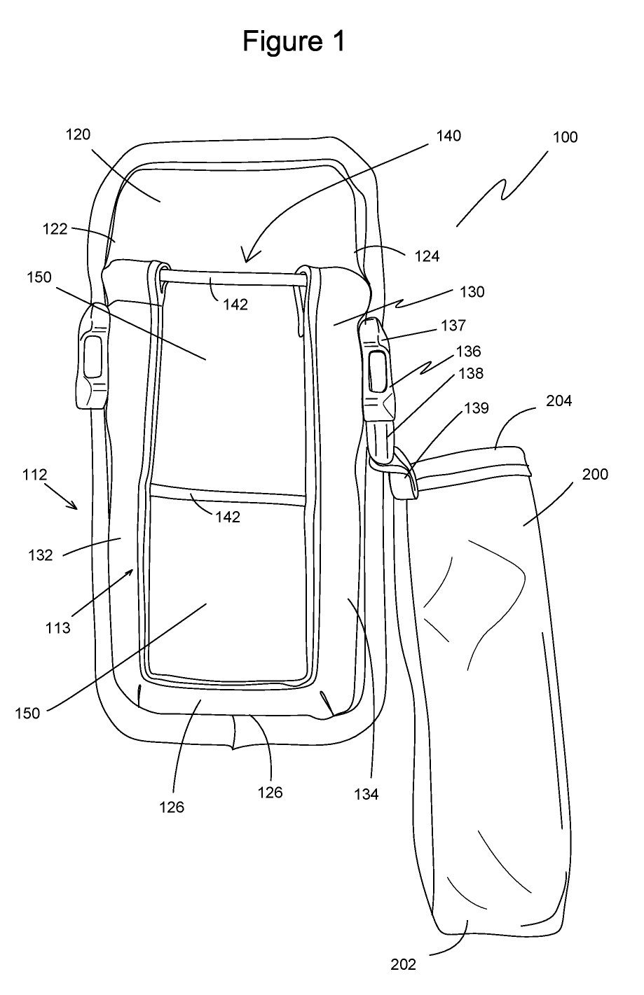

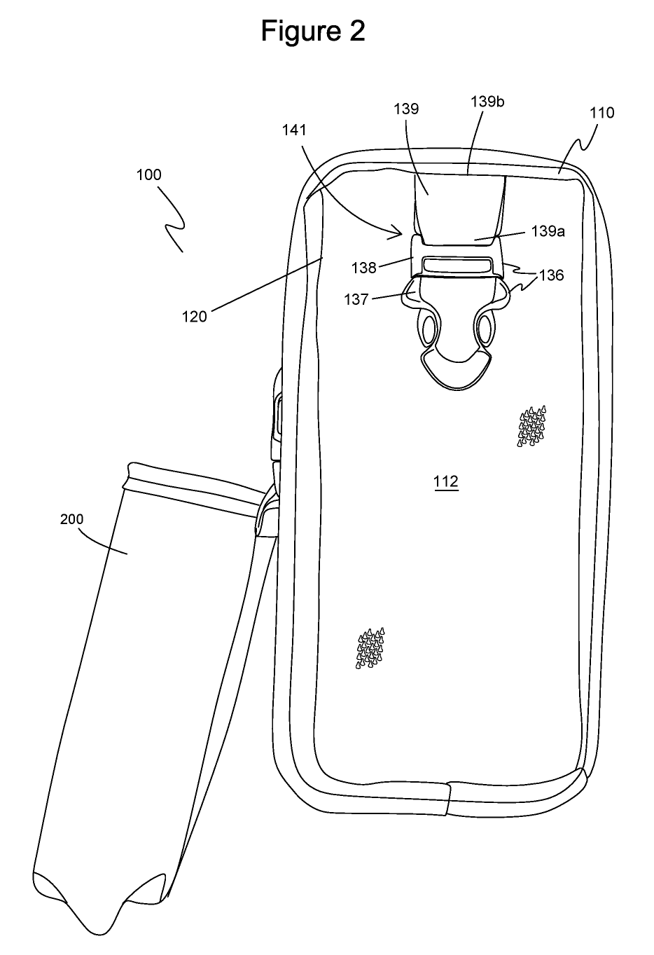

The preferred embodiment(s) of the present invention is illustrated in FIGS. 1-8. FIG. 1 shows a front view of one embodiment of a tool pouch assembly 100 constructed, for example, to retain a portable gas detector 5 (shown in FIG. 2) and a sensor probe 10. The tool pouch assembly 100 includes a tool pouch 110 and a probe pocket 200 that is releasably attached to the tool pouch 110. The probe pocket 200 is constructed to receive the sensor probe 10 of the portable gas detector 5.

The tool pouch 110 has a sidewall extending up from a closed bottom end portion 113 with a first side portion 132, a second side portion 134, a front portion 135, and a back portion 112. In one embodiment shown in FIG. 1, the back portion 112 of the tool pouch 110 is a generally-planar support panel 120. The pouch body portion 130 is attached to the support panel 120 to define a storage compartment 140 sized to receive the gas detector 5 or other instrument, meter, or tool. In one embodiment, the support panel 120 includes a substrate made of cardboard, plastic, leather, or other rigid or semi-rigid material, where the substrate is encased in fabric or protective material. For example, the substrate is a plastic plate encased in a woven nylon material.

The pouch body portion 130 has a first side portion 132, a second side portion 134, and a bottom portion 136 that each attach to and extend in a spaced-apart relationship from the respective panel first side portion 122, panel second side portion 124, and panel bottom portion 126. In one embodiment, the first side portion 132 and the second side portion 134 of the pouch body portion 130 are spaced from each other and define an open area 150 between them along the front portion 113 of the tool pouch 110. The open area 150 is useful for viewing a display or visual alerts on the gas detector 5 as well as for accessing controls on the gas detector 5 or other instruments stored in the storage compartment 140. In one embodiment, one or more connecting bands 142 extend across the open area 150 and are connected to the first and second side portions 132, 134. In some embodiments, the connecting bands 142 are made of a stretchable material, such as elasticized cord or the like. When the connecting band(s) 142 are made with a stretchable material, the storage compartment 140 is expandable to facilitate receiving the gas detector 5 while also resiliently returning to smaller state to provide a snug fit on the gas detector 5.

One or more connector first portion 137 of a connector 136 is secured to the pouch body portion 130, such as along the first side portion 132 and/or second side portion 134. A connector second portion 138 on the probe pocket 200 or other accessory mates with and engages the connector first portion 137 to releasably attach the probe pocket 200 or other accessory to the tool pouch 110. The connector first portion 137 is positioned on the pouch body to engage the connector second portion. For example, the connector first portion 137 and connector second portion 138 are the female and male parts, respectively, of a side release buckle as commonly used with webbing. Other forms of the connector 136 are contemplated, including one or more snaps, a hook-and-loop fastener, a spring hook, a strap with rings or buckle, MOLLE webbing attachment system, and the like.

As shown in FIG. 1, probe pocket 200 is an accessory that is releasably attached to the tool pouch 110 using the connector 136, where the second connector portion 138 is secured to the probe pocket with webbing 139. In other embodiments, the probe pocket 200 is fixedly secured to the tool pouch 110 and is not removable except by partial disassembly or partial destruction of the pouch assembly 100. For example, the probe pocket 200 is fixedly attached to the tool pouch 110 by stitching or a mechanical fastener.

In one embodiment, the probe pocket 200 has an elongated, tubular shape that extends from a closed bottom end 202 to an open top end. As shown, the probe pocket 200 is constructed and sized to receive a sensor probe 10 of the portable gas detector 5. The cross-sectional shape of the probe pocket 200 may be circular, rectangular, or other shape as desired and appropriate for the equipment to be stored. In one embodiment, the probe pocket 200 is made of a pliable material, such as nylon, canvas, neoprene, vinyl, or leather. In other embodiments, the probe pocket 200 is made at least in part of a mesh or otherwise defines openings to allow gases to flow through the probe pocket 200 to reach the sensor probe 10 when it is stored in the probe pocket 200.

Turning now to FIG. 2, a rear view of the pouch assembly 100 shows a back surface 112 of the tool pouch 110 with a connector 136 and webbing 139. The connector 136 is shown in a closed position or storage position with the connector second portion 138 engaging the connector first portion 137. The connector first portion 137 is fixedly attached to the support panel 120 and the connector second portion 138 is attached to the webbing 139. The webbing 139 has a first webbing end 139a attached to the connector second portion 138 and a second webbing end 139b secured to the support panel 120. The connector 136, webbing 139, and support panel 120 define a closed loop 141 that may be used to attach the tool pouch 110 to a D-ring or other structure. For example, after passing the connector second portion 138 through a D-ring, the connector second portion 138 engages the connector first portion 137 to link the D-ring to the loop 141. Alternately, the connector second portion 138 of the tool pouch 110 may engage a different connector first portion 137′ (not shown) located on a harness, tool belt, backpack, or other location in order to attach the tool pouch 110 to that object. In such embodiments, the connector first portion 137 on the tool pouch 110 may be used to stow the connector second portion 138 when not in use.

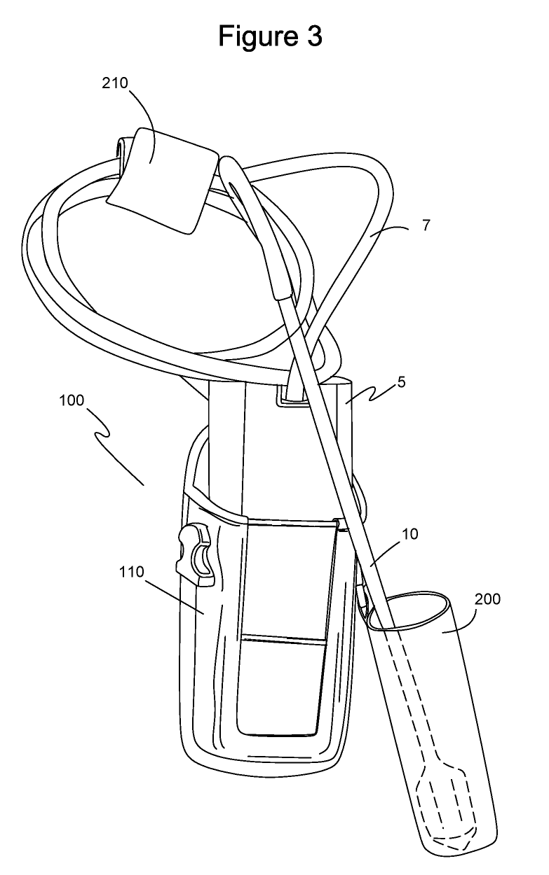

Turning now to FIG. 3, a front perspective view shows another embodiment of the pouch assembly 100 with a portable gas detector 5. The gas detector 5 is retained in the storage compartment 146 of the tool pouch 110. A length of tubing 7 extends from the gas detector 5 to the sensor probe 10, which is stored in the probe pocket 200. The probe pocket 200 defines a plurality of through openings 217 in the sidewall to allow the sensor probe 10 to detect gases while in the probe pocket 200. Through openings 217 may additionally or alternately be defined in the closed bottom end 202. A cord-lock 210, such as a strap with hook-and-loop fasteners, is installed in a closed loop around a coil of the tubing 7 and fastened to itself. As shown, both the first side portion 132 and the second side portion 134 of the tool pouch 110 have a connector first portion 137. Accordingly, the probe pocket 200 may be attached to either side of the tool pouch 110 as desired. In other embodiments, the tool pouch 110 includes two probe pockets 200 or other accessories that are attached to the tool pouch 110 using connectors 136.

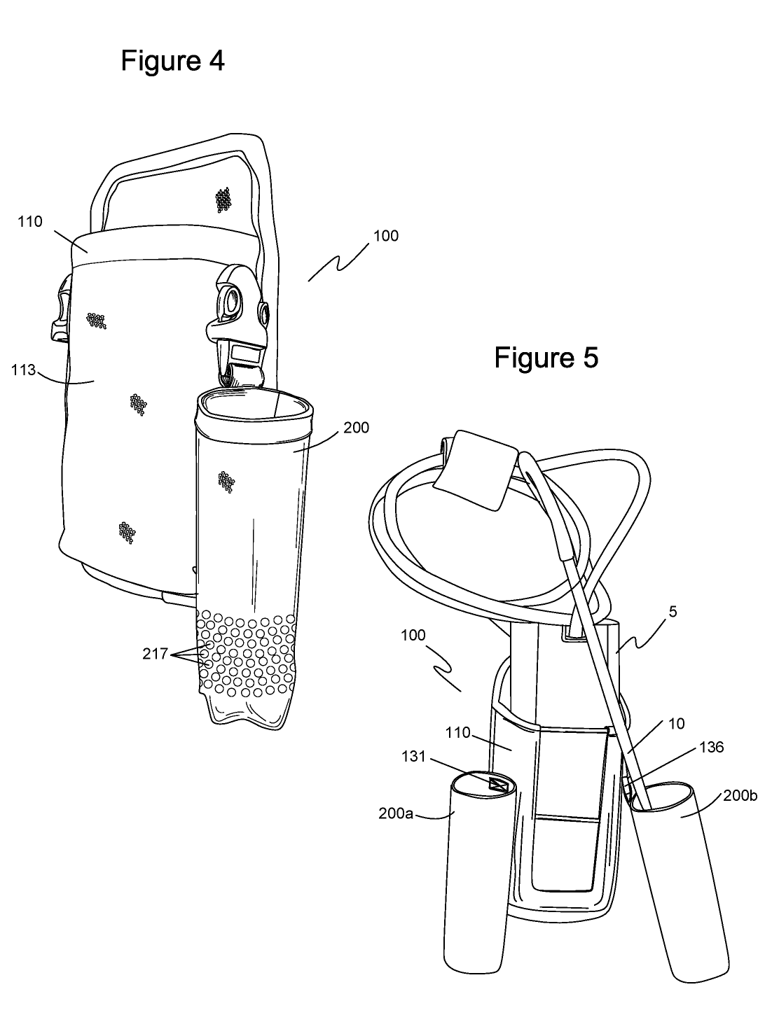

Turning now to FIGS. 4 and 5, additional embodiments of the tool pouch assembly 100 are illustrated in front perspective views. The tool pouch 110 of FIG. 4 has a closed front portion 113 rather than defining an open region 150 as shown in FIG. 1. The probe pocket 200 defines a plurality of through openings 217. Through openings 217 enable the sensor probe 10 (shown in FIG. 5) to better sample gases while the sensor probe 10 is disposed in the probe pocket 200. The tool pouch of FIG. 5 has a first probe pocket 200a permanently attached by stitching 131 to the tool pouch 110 and a second probe pocket 200b releasably attached to the tool pouch 110 by the connector 136.

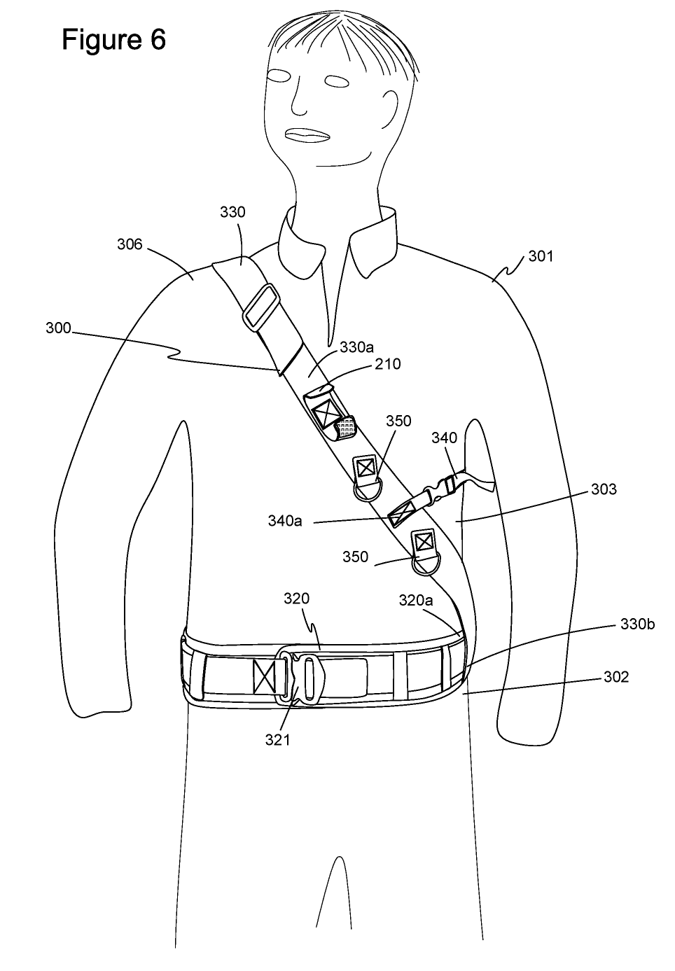

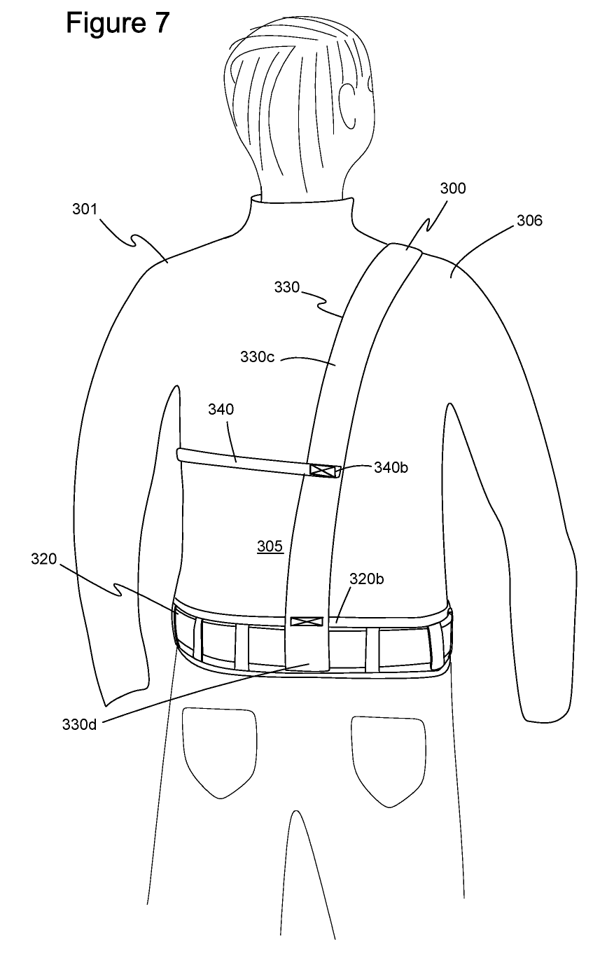

Turning now to FIG. 6, a front elevational view shows one embodiment of a body harness 300 of the present invention as donned by a user 301. FIG. 7 shows a rear view of the body harness 300 as donned by the user 301. The body harness 300 has a waist belt 320 configured to form a closed loop around a waist 302 of the user 301. The waist belt 320 has a side portion 320a corresponding to the user's side 303 and a rear portion 320b corresponding to the user's back 305 (shown in FIG. 7). Preferably, the waist belt 320 is adjustable and has a buckle or other fastening device or closure 321. In some embodiments, the waist belt 320 is optionally padded for comfort.

The body harness 300 also has a shoulder strap 330 that includes a front portion 330a with a first strap end 330b and a back portion 330c with a second strap end 330d (shown in FIG. 7). The shoulder strap 330 is constructed to extend over the user's shoulder 306, diagonally across the front of the user's torso, and down the user's back 305 with the first strap end 330b connected to the side portion 320a of the waist belt 320 and the second strap end 330d connected to the rear portion 320b of the waist belt 320. In some embodiments, the first strap end 330b and/or the second strap end 330d are fixedly attached (e.g., by stitching, rivets, etc.) to the waist belt 320 so as not to be removed by the user without disassembly or partial destruction of the body harness 300. For example, the second strap end 330d is stitched to itself in a loop that encircles the waist belt 320 or a component thereof, is stitched directly to the waist belt 320, or is mechanically fastened to the waist belt 320. In other embodiments, the first strap end 330b and/or the second strap end 330d are removably attached to the waist belt 320, such as by a side release buckle, slider buckle, or other connection hardware.

The body harness 300 also has a side strap 340 connected to and extending between the front portion 330a and the back portion 330c of the shoulder strap 330. A first side-strap end 340a is connected to the front portion 330a of the shoulder strap 330. A second side-strap end 340b is connected to the back portion 330c of the shoulder strap 330. When worn by the user 301, the side strap 340 is intended to extend horizontally around the user's side 303, where the side strap 340 is vertically spaced from and generally parallel to the side portion 320a of the waist belt 320. In some embodiments, the side strap 340 has an adjustable length. In other embodiments, the side strap 340 is made of a stretchable material, such as elasticized nylon. The first and/or second side-strap ends 340a, 340b may be fixedly attached or releasably attached to the shoulder strap 330. For example, first and second side-strap ends 340a, 340b are secured to the shoulder strap 330 by stitching or mechanical fasteners. The side strap 340 advantageously maintains the shoulder strap 330 from slipping off the user's shoulder 306. Another advantage of the side strap 340 is distributing gravitational forces of equipment attached to the front portion of the shoulder strap 330 so as to resist pulling down on the front portion 330a of the shoulder strap 330 and resist pulling up on the back portion 330c of the shoulder strap 330. Yet another advantage of the side strap 340 is that it maintains the shoulder strap 330 close to the user's body when the user is climbing to prevent a swinging motion of equipment attached to the shoulder strap 306 as occurs with pouches attached to the hip of one's tool belt.

Some embodiments of harness include one or more anchor points 350 for accessories and pouches. For example, anchor points 350 are D-rings, hooks, fasteners, or connectors 136 (or part thereof). In one embodiment, harness 300 includes two D-ring anchor points 350 on the front portion 330a of the shoulder strap 330 for attaching the pouch assembly 110 discussed above or other storage pouch. In one embodiment, harness includes an optional cord-lock 210 strap secured to the front portion 330a of the shoulder strap. For example, the cord-lock 210 is a strap that may be closed around a coil of tubing 7, a length of cord, or other object. In one embodiment, D-ring anchor points 350 are attached to the front portion 330a of the shoulder strap 330, such as adjacent the side portion 320a of the waist belt 320. When positioned as described and shown in FIG. 6, embodiments of the pouch assembly 100 are conveniently located to be accessible, yet out of the way of the user 301 when used to hold the portable gas detector 5.

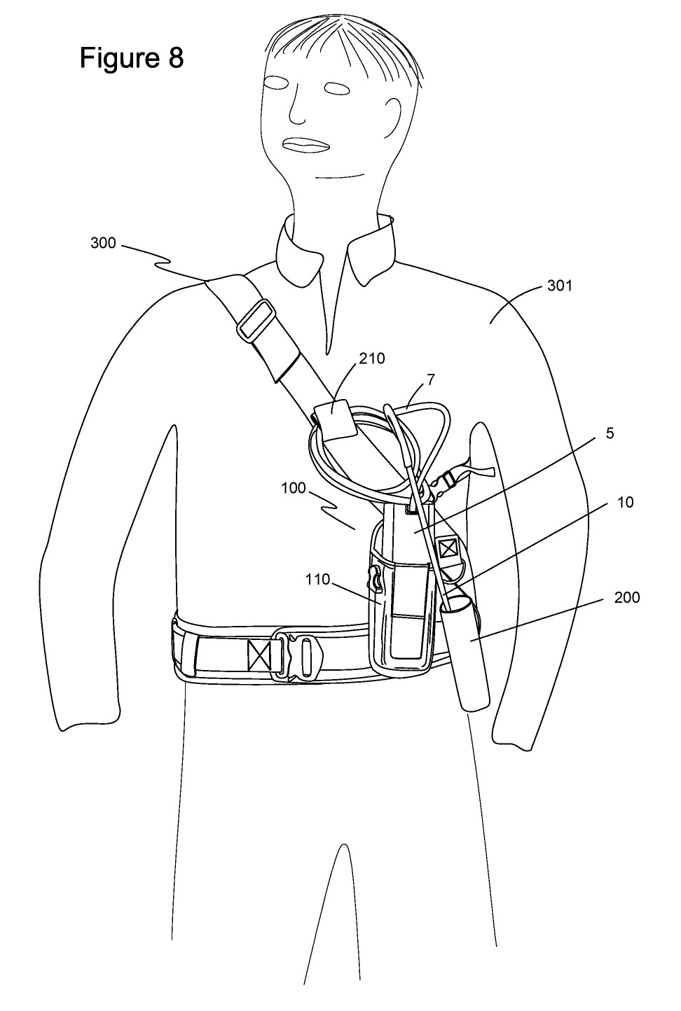

Referring now to FIG. 8, a front view of the user 301 illustrates one embodiment of the body harness 300 in use with an embodiment of the pouch assembly 100 attached to the body harness 300. The pouch assembly 100 is attached to the harness 300 using the connector 136 and webbing 139 on the back surface 112 of the support panel 120 (shown in FIG. 2), where the webbing 139 extends through the D-ring of one anchor point 350. The probe pocket 200 houses the sensor probe 10 and is attached to the pouch 110 as discussed above. The cord lock 210 strap on the harness 300 engages a coil of tubing 7 of the portable gas detector 5 to maintain the tubing 7 in a stowed position.

In use, embodiments of the body harness 300 and pouch assembly 100 are useful for working with portable gas detectors 5, radios, electrical test equipment, dictation equipment, and other equipment used by facilities maintenance personnel. Embodiments of the body harness 300 allow for comfortable and convenient placement of the pouch assembly 100 in addition to accepting other tool pouches as desired. The probe pocket 200 provides a convenient storage location of the sensor probe 10, a microphone, test probes, and other equipment connected to an instrument or device retained in the storage compartment 140 of the tool pouch 110 while the user is climbing or working in restricted areas. For example, the user may temporarily use the sensor probe 10 and then replace it into the probe pocket 200 while climbing or performing other tasks. The cord lock 210 on the harness retains tubing 7 and cords out of the way of the user, yet allows the user to release the tubing 7 as needed.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

We claim:

1. A tool pouch comprising:
a support panel having a front face, a back face, a panel first side portion, a panel second side portion, and a panel bottom portion;
a pouch body portion having a first side portion, a second side portion spaced apart from and generally opposing the first side portion, and a bottom portion, wherein the pouch body portion is attached to the support panel along the respective panel first side portion, the panel second side portion, and the panel bottom portion and extends out from the front face of the support panel to define a storage compartment, and wherein the pouch body portion defines an open region between the first side portion, the second side portion, and the bottom portion;
one or more straps extending across the open region and connecting the first side portion to the second side portion; and
a connector first portion fixedly secured to at least one of the first side portion and the second side portion of the pouch body portion, wherein the connector first portion is positioned on an outside surface of the pouch body portion to releasably engage a corresponding connector second portion.

2. The tool pouch of claim 1, each of the one or more straps is convertible between a larger state and a smaller state.

3. The tool pouch of claim 2, wherein each of the one or more straps is made of a stretchable material.

4. The tool pouch of claim 1, further comprising:
a probe pocket made of a pliable material and having a pocket sidewall with an elongated tubular shape extending from a closed bottom end to an open top end, and having the connector second portion secured to the probe pocket adjacent the open top end, wherein the connector second portion is constructed to releasably engage the connector first portion to releasably attach the probe pocket to the main pouch body.

5. A tool pouch comprising:
a main pouch body having a closed bottom end portion and a sidewall extending up from the closed bottom end portion with a first side portion, a second side portion spaced apart from and generally opposing the first side portion, a front portion, a rear portion spaced apart from and generally opposing the front portion, wherein the pouch body defines a storage compartment;
a connector first portion fixedly secured to the sidewall and positioned on an outside surface of the pouch body; and
a probe pocket made of a pliable material and having a pocket sidewall with an elongated tubular shape extending from a closed bottom end to an open top end, and having a connector second portion secured to the probe pocket adjacent the open top end, wherein the connector second portion operably engages the connector first portion to releasably attach the probe pocket to the main pouch body.

6. The tool pouch of claim 5, wherein the front portion of the main pouch body defines an open region between the first side portion and the second side portion, and wherein the tool pouch further comprises one or more straps extending across the open region and connecting the first side portion to the second side portion.

7. The tool pouch of claim 6, wherein each of the one or more straps is convertible between a larger state and a smaller state.

8. The tool pouch of claim 7, wherein each of the one or more straps is made of a stretchable material.

9. The tool pouch of claim 5, wherein the probe pocket defines a plurality of through openings in at least one of the pocket sidewall and the closed bottom end.

10. A body harness comprising:
a waist belt configured to form a closed loop around a user's waist and having a side portion corresponding to the user's side, a rear portion corresponding to the user's back, and a front portion with a closure;
a shoulder strap constructed to extend over an user's shoulder and having a front portion with a first strap end and a back portion with a second strap end, wherein the first strap end is connected to the side portion of the waist belt and the second strap end is connected to the rear portion of the waist belt; and
a side strap connected to and extending between the front portion and the back portion of the shoulder strap, wherein the side strap is vertically spaced from and extends generally parallel to the side portion of the waist belt.

11. The body harness of claim 10 further comprising:
a flexible cord-lock strap secured to the body harness and having a releasable closure configured to releasably fasten the cord-lock strap in a closed loop.

12. The body harness of claim 10 further comprising one or more anchor point with a feature selected from the group consisting of a closed-loop connector, a releasable connector, and a cord loop.

13. The body harness of claim 12 further comprising:
a tool pouch comprising:
a main pouch body having a closed bottom end portion and a sidewall extending up from the closed bottom end portion to define a storage compartment, the sidewall having a first side portion, a second side portion spaced apart from and generally opposing the first side portion, a front portion, a rear portion spaced apart from and generally opposing the front portion;
a connector first portion fixedly secured to the sidewall and positioned on an outside surface of the pouch body; and
a selectively-closable attachment loop secured to the rear portion and sized to extend through the closed-loop connector, the releasable connector, or the cord loop on the anchor point.

14. The body harness of claim 13 further comprising:
a probe pocket made of a pliable material and having a pocket sidewall with an elongated tubular shape extending from a closed bottom end to an open top end, and having a connector second portion secured to the probe pocket adjacent the open top end, wherein the connector second portion is constructed to operably mate with the connector first portion to releasably secure the probe pocket to the main pouch body.

15. The body harness of claim 10 further comprising:
a tool pouch releasably attached to the body harness and comprising:
a support panel having a front face, a back face, a panel first side portion, a panel second side portion, and a panel bottom portion;
a pouch body portion having a first side portion, a second side portion spaced apart from and generally opposing the first side portion, and a bottom portion, wherein the pouch body portion is attached to the support panel along the respective panel first side portion, the panel second side portion, and the panel bottom portion and extends out from the front face of the support panel to define a storage compartment, and wherein the pouch body portion defines an open region between the first side portion, the second side portion, and the bottom portion;
one or more straps extending across the open region and connecting the first side portion to the second side portion; and
a connector first portion fixedly secured to at least one of the first side portion and the second side portion of the pouch body portion, wherein the connector first portion is positioned on an outside surface of the pouch body portion to releasably engage a corresponding connector second portion.

16. The body harness of claim 15, wherein each of the one or more straps is convertible between a larger state and a smaller state.

17. The tool pouch of claim 16, wherein each of the one or more straps is made of a stretchable material.

18. The tool pouch of claim 15, wherein the pocket sidewall of the probe pocket defines a plurality of through openings.
